(19) United States
(12) Patent Application Publication
(10) Pub. No.: US 2008/0294270 A1
(43) Pub. Date: Nov. 27, 2008

(54) DIFFERENTIALLY PROCESSED TISSUE AND PROCESSING METHODS THEREOF

(75) Inventors: Jian Q. Yao, Austin, TX (US); Hai-Qing Xian, Austin, TX (US); Jizong Gao, Cedar Park, TX (US); Rodney Bristol, Cedar Park, TX (US); Victor Zaporojan, Austin, TX (US); Hali Wang, The Hills, TX (US); Hui Liu, Austin, TX (US)

Correspondence Address: WOOD, HERRON & EVANS, LLP (ZIMMER), 2700 CAREW TOWER, 441 VINE STREET, CINCINNATI, OH, 45202, US

(73) Assignee: ZIMMER ORTHOBIOLOGICS, INC., Austin, TX (US)

(21) Appl. No.: 11/753,102

(22) Filed: May 24, 2007

(51) Int. Cl. A61L 27/38; A61F 2/28; D01C 3/00

(52) U.S. Cl. 623/23.63; 623/1.41; 623/16.11; 8/94.11

(57) ABSTRACT

A method of preparing an implantable biological device having a first tissue part and a second tissue part includes exposing the first tissue part to a first preparation method and preventing exposure of the second biological tissue part to the first preparation method. Preventing exposure of the second biological tissue part to the first preparation method may be achieved using an embedding technique, a coating technique, a covering technique, or physical isolation. The method may further include exposing the second tissue part to a second preparation method and preventing exposure of the first biological tissue part to the second preparation method. An apparatus for preparing an implantable biologic device includes an enclosure having first and second chambers separated by a partition member wherein a substantial portion of the first tissue part is within the first chamber and a substantial portion of the second tissue part is within the second chamber.

### TECHNICAL FIELD

Apparatus and method for preparing tissue implants and devices, more particularly apparatus and method for processing tissue parts of an implant differently.

### BACKGROUND

Various orthopedic procedures include replacing damaged native tissue with a tissue implant or device procured from another source. For instance, in a wide variety of procedures, autografts, allografts, and/or xenografts may be used to affect repair, replacement, or augmentation of damaged or otherwise non-functioning tissue within a patient. Prior to use within a patient, however, the tissue implant will typically be treated or processed to make the implant more suitable for implantation into the patient. In this regard, tissue implants may be subjected to processing to remove undesired tissue parts or components while maintaining desired tissue parts or components. For example, a tissue implant may be treated to remove fat and bone marrow tissue while maintaining the bone portion of the implant. The preparation of the tissue implants may be achieved through various processes including physical scrubbing, chemical treatment, or other processes known in the art.

One drawback of current processes for preparing tissue implants is that the processes are directed primarily to the preparation of homogeneous tissue implants, i.e., implants having a single type of tissue. In other words, the current processes maintain the desired tissue part but remove or effectively eliminate the functional aspects of other tissue parts surrounding the desired tissue part. Many orthopedic tissues, however, are inhomogeneous and have multiple tissue parts with distinctive physical, chemical, and biological properties, and which are desirable for certain types of tissue implants. Examples of such inhomogeneous tissue parts include bone-tendon-bone, osteochondral plugs, the meniscus with its red and white zones, and others. Treatment of inhomogeneous tissue parts using current processes destroy or severely diminish the unique properties of the tissue parts for which the processing method is not directed. By way of example, processing bone parts to remove fat and bone marrow tissue may diminish the usefulness of cartilage and tendons associated with the bone parts, even though the cartilage and tissue may be desired for the tissue implant.

Therefore, there is a need for improvements in a method and apparatus for preparing an inhomogeneous tissue implant so as to preserve the unique properties of multiple tissue parts.

### SUMMARY

Method and apparatus for differentially processing multiple tissue parts to preserve the properties of each of the tissue parts. In one embodiment, a method of preparing an implantable biological device having at least a first biological tissue part and a second biological tissue part includes exposing the first biological tissue part to a first preparation method and simultaneously substantially preventing exposure of the second biological tissue part to the first preparation method. In this embodiment, such is achieved by embedding the second biological tissue part in a material that substantially prevents exposure of the second biological tissue part to the first preparation method. The embedding material may include a gas, liquid, solid and/or a semi-solid. More particularly and without limitation, the embedding material may include wax, polystyrene, poly(methyl methacrylate), poly-alginate, agarose gel, combinations of these materials, and other materials. The method may further include exposing the second biological tissue part to a second preparation method, and in addition simultaneously substantially preventing exposure of the first biological tissue part to the second preparation method by embedding the first biological tissue part in a material that substantially prevents exposure of the first biological tissue part to the second preparation method. In one embodiment, the first tissue part is bone and the second tissue part may be cartilage, tendon, ligament, meniscus, periosteum, muscle, and/or other tissue parts. In another embodiment, the first and second tissue parts may be different sections of a homogeneous tissue.

In another embodiment, a method of preparing an implantable biological device having at least a first biological tissue part and a second biological tissue part includes exposing the first biological tissue part to a first preparation method and simultaneously substantially preventing exposure of the second biological tissue part to the first preparation method. In this embodiment, such is achieved by coating the second biological tissue part with at least one material that substantially prevents exposure of the second biological tissue part to the first preparation method. The coating material may include without limitation wax, polystyrene, poly(methyl methacrylate), combinations of these materials, and other materials. The method may further include exposing the second biological tissue part to a second preparation method, and in addition simultaneously substantially preventing exposure of the first biological tissue part to the second preparation method by coating the first biological tissue part in a material that substantially prevents exposure of the first biological tissue part to the second preparation method. In one embodiment, the first tissue part is bone and the second tissue part may be cartilage, tendon, ligament, meniscus, periosteum, muscle, and/or other tissue parts. In another embodiment, the first and second tissue parts may be different sections of a homogeneous tissue. In addition, the coating(s) may be selectively permeable to at least one component of a reagent used in the first and/or second preparation method.

In another embodiment, a method of preparing an implantable biological device having at least a first biological tissue part and a second biological tissue part includes exposing the first biological tissue part to a first preparation method and simultaneously substantially preventing exposure of the second biological tissue part to the first preparation method. In this embodiment, such is achieved by covering the second biological tissue part with a cover that substantially prevents exposure of the second biological tissue part to the first preparation method.

In one embodiment, the cover may include a first open end and a second closed end to define an interior cavity. The second biological tissue part may be disposed in the interior cavity of the cover and the first end of the cover sealed to substantially enclose the second tissue part inside the cover. The method may further include maintaining at least one preparation solution in the interior cavity of the cover. The cover may be selectively permeable to at least one component of a reagent(s) used in the first preparation method, or may be impermeable to reagent(s) used in the first preparation method.

In an alternate embodiment, the cover may include a first open end and a second open end to define an interior passage. The second biological tissue part may be disposed in the interior passage of the cover and the first and second ends of the cover sealed to substantially enclose the second tissue part inside the cover. The method may further include maintaining at least one preparation solution in the interior passage of the cover. The cover may be selectively permeable to at least one component of a reagent used in the first preparation method or be impermeable to the reagent used in the first preparation method. In the embodiments employing a cover, the first tissue part may be bone and the second tissue part may be cartilage, tendon, ligament, meniscus, periosteum, muscle, and/or other tissue parts. Alternatively, the first and second tissue parts may be different components of a homogeneous tissue. In addition, the method may further include exposing the second biological tissue part to a second preparation method, and in addition simultaneously substantially preventing exposure of the first biological tissue part to the second preparation method by covering the first biological tissue part in a material that substantially prevents exposure of the first biological tissue part to the second preparation method.

In another embodiment, a method of preparing an implantable biological device having at least a first biological tissue part and a second biological tissue part includes exposing the first biological tissue part to a first preparation method and simultaneously substantially preventing exposure of the second biological tissue part to the first preparation method. In this embodiment, such is achieved by positioning the first biological tissue part in a first chamber of an enclosure, and positioning the second biological tissue part in a second chamber of the enclosure that is substantially isolated from the first chamber. For example, the first biological tissue part may be exposed to a first processing reagent within the first chamber of the enclosure and the second biological tissue part may be exposed to a second processing reagent within the second chamber of the enclosure. Moreover, the temperature, pressure, and/or the concentration of the first and/or second reagents may be varied or otherwise manipulated in the respective first and/or second preparation methods. When temperature is manipulated in the first and/or second preparation methods, the first and second chambers may be thermally isolated from each other. In one embodiment, the first tissue part may be bone and the second tissue part may be cartilage, tendon, ligament, meniscus, periosteum, muscle, and/or other tissue parts. In another embodiment, the first and second tissue parts may be different components of a homogeneous tissue.

In one embodiment, an apparatus for preparing an implantable biologic device in a manner previously described includes an enclosure having a first chamber and a second chamber separated by a partition member that substantially isolates the chambers from one another. The partition member includes at least one aperture adapted to receive an implantable biological tissue having a first tissue part and a second tissue part. The biological tissue is received in the aperture so that a substantial portion of the first tissue part is within the first chamber and a substantial portion of the second tissue part is within the second chamber. Each of the first and second chambers includes an inlet and an outlet for exposing the respective tissue parts to at least one processing reagent, which may be the same or different for each chamber. The apparatus may further include a thermal control unit coupled to one or both chambers for controlling the temperature of the first and/or second reagent(s). A pump may also be operatively coupled to one or both chambers for controlling the pressure of the first and/or second reagent(s). The apparatus may further include valves for controlling the flow rate of the processing reagent(s) through the chambers.

In addition to exposing the tissue parts to reagents, the first and/or second preparation methods may include other processing steps. By way of example and without limitation, the first and/or second preparation methods may include agitation (e.g., shaker, vortex, tilt table), exposure to light/dark conditions (e.g., exposure to UV light), chemical crosslinking and/or other standard tissue processing steps.

These and other embodiments will become more readily apparent to those of ordinary skill in the art upon review of the following detailed description taken in conjunction with the accompanying drawings.

### BRIEF DESCRIPTION OF THE DRAWINGS

The accompanying drawings, which are incorporated in and constitute a part of this specification, illustrate embodiments of the invention and, together with the summary given above, and the detailed description given below, serve to explain the invention.

FIG. 1 is a front view of a schematic exemplary tissue implant in accordance with some embodiments;

FIG. 1A is an exemplary algorithm for preparing a tissue implant in accordance with some embodiments;

FIG. 2A is an cross-sectional view of an apparatus illustrating protecting the second tissue part from exposure to the first preparation method by embedding in accordance with one embodiment;

FIG. 2B is an cross-sectional view similar to FIG. 2A illustrating protecting the first tissue part from exposure to the second preparation method by embedding;

FIG. 3A is an cross-sectional view of an apparatus illustrating protecting the second tissue part from exposure to the first preparation method by coating in accordance with one embodiment;

FIG. 3B is an cross-sectional view similar to FIG. 3A illustrating protecting the first tissue part from exposure to the second preparation method by coating;

FIG. 4A is an cross-sectional view of an apparatus illustrating protecting the second tissue part from exposure to the first preparation method by covering in accordance with one embodiment;

FIG. 4B is an cross-sectional view similar to FIG. 4A illustrating protecting the first tissue part from exposure to the second preparation method by covering;

FIG. 5A is an cross-sectional view of an apparatus illustrating protecting the second tissue part from exposure to the first preparation method by covering in accordance with one embodiment;

FIG. 5B is an cross-sectional view similar to FIG. 5A illustrating protecting the first tissue part from exposure to the second preparation method by covering;

FIG. 6 is a cross-sectional view of an apparatus illustrating protecting the first and second tissue parts from exposure to the first and second preparation methods by physical isolation in accordance with one embodiment.

### DETAILED DESCRIPTION

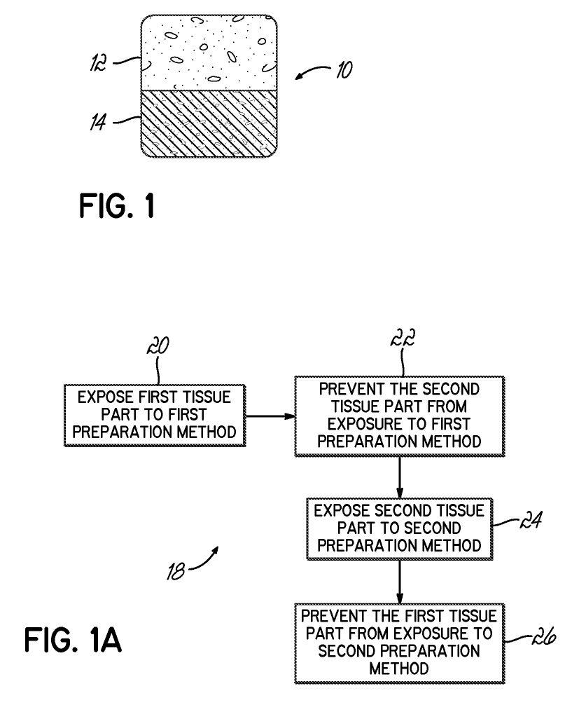

Embodiments include methods and apparatus for preparing or processing an implantable biologic device having multiple tissue parts. For example, FIG. 1 schematically illustrates a tissue implant 10 having at least a first biological tissue part 12 and a second biological tissue part 14. The tissue implant 10 may include any type of medical implant for affecting treatment in the body of a patient having at least one tissue type associated therewith. Thus, the implant 10 may include at least one tissue obtained from autografts, allografts, and/or xenografts, and may be used in a wide range of surgical procedures, including various orthopedic procedures. The aforementioned applications are exemplary and not limiting, and may provide benefits for other biological devices utilizing one or multiple tissue parts. The first and second tissue parts may include a wide range of biological tissues including and without limitation, bone, cartilage, tendon, ligament, meniscus, periosteum, and/or muscle, and other tissue such as spinal, neural, or dental tissue. Moreover, while the tissue implant 10 is shown and described as inhomogeneous having first and second tissue parts that consist of different tissue types (e.g., bone versus cartilage), the tissue implant 10 may alternately be homogeneous wherein the first and second tissue parts are sections of the same tissue (e.g., different portions of the same bone, or same cartilage, etc.). Thus, embodiments are not limited to the preparation of inhomogeneous tissue, but may also be used in the preparation of homogeneous tissue where there is a need or desire to treat multiple sections of the same tissue differently.

In addition, the tissue implants are not limited to consisting of only biologic tissue, but may include artificial or synthetic components as well. Thus, a tissue implant as used herein may encompass not only natural/biologic implants but also hybrid implants having both biologic tissue and other artificial/synthetic components. By way of example, one such hybrid implant, as disclosed in WO 2007/025290 and assigned to the assignee of the present application, includes a tissue graft coupled to or grown on an artificial porous surface. Another hybrid implant may include a synthetic bone material, such as calcium phosphate, calcium sulfate, or the like, coupled to a soft tissue graft, such as tendon or ligament, to serve as an anchor.

The embodiments described below generally follow a similar algorithm for preparing the tissue implant. FIG. 1A shows an exemplary general algorithm 18 employed, in whole or in part, in the various embodiments. This algorithm 18 includes exposing a first tissue part to a first preparation method as at 20, and simultaneously substantially preventing exposure of a second biological tissue part to the first preparation method as at 22. The algorithm 18 may additionally include exposing the second tissue part to a second preparation method as at 24, and simultaneously substantially preventing exposure of the first biological tissue part to the second preparation method as at 26. One difference in the various embodiments described below is the manner in which the biological tissue parts are protected (e.g., via isolating, covering, coating and/or embedding) from the preparation methods used in processing the tissue implant. Advantageously, differential processing as described herein can maintain specific, unique, and/or beneficial properties of each tissue part (e.g., without compromising and/or altering the other part's properties).

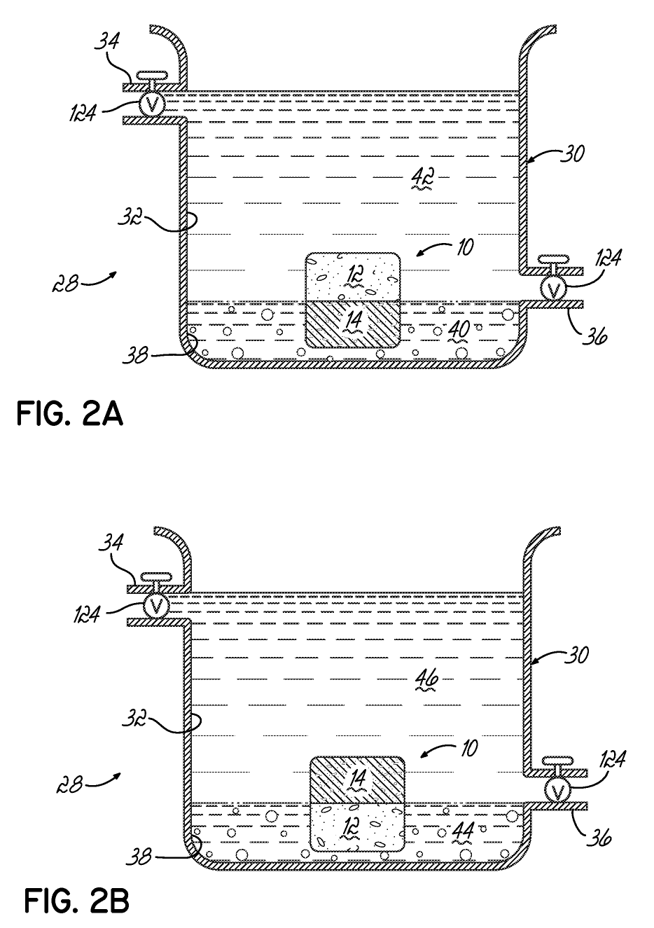

FIGS. 2A and 2B illustrate one embodiment wherein the tissue parts 12, 14 of a tissue implant 10 are substantially protected from the preparation methods through an embedding technique. To this end, an apparatus 28 for preparing a tissue implant 10 includes a container 30 having an interior cavity 32 and a fluid inlet 34 and outlet 36 in communication with the interior cavity 32. The container 30 may optionally include a top (not shown) for closing off the interior cavity 32. As shown in FIG. 2A, the bottom portion 38 of the cavity 32 contains an embedding material 40 into which the second tissue part 14 is embedded and from which the first tissue part 12 extends. As shown in FIG. 2A, the outlet 36 to the cavity 32 is positioned above the embedding material 40.

In one embodiment, the embedding material 40 is a solid or semi-solid material such as, for example, wax, polystyrene, poly(methyl methacrylate), poly-alginate, agarose gel, combinations thereof, and other materials known to those of ordinary skill in the art. In addition, the embedding material 40 is not limited to a solid or semi-solid material but may also include a fluid (e.g., liquid or a gas). For example, one part of the tissue may be immersed in a liquid while the other part may be in another liquid that is immiscible with the first liquid (e.g., the first liquid is polar and the second is non-polar). As explained below, in some embodiments the embedding material 40 may be substantially non-reactive with the second tissue part 14 and which substantially prevents the second tissue part 14 from exposure to the first preparation method. It also may be desirable to employ an embedding material 40 that is relatively easy to remove (e.g., by physical or chemical means) from the second tissue part 14.

With the second tissue part 14 embedded in the material 40 and the first tissue part 12 positioned within the cavity 32, the first tissue part 12 may be processed using a first preparation method. For example, a processing reagent 42, such as acidic solution, base solution, detergents, organic solvent and enzyme solution, etc., may flow through the cavity 32 via the fluid inlet and outlet 34, 36, respectively, so as to treat the first tissue part 12. While container 30 includes an inlet and outlet that permits a fluid to flow through the cavity 32, the invention is not so limited as the inlet and outlet may be omitted and the container 30 adapted to hold or contain processing reagent 42 without flow-through capability. As noted above, the embedding material 40 protects the second tissue part 14 and substantially prevents exposure of the second tissue part 14 to the first preparation method. Thus, for example, the processing reagent 42 is not capable of substantially penetrating the embedding material 40 so as to reach and/or react with the second tissue part 14. After the first tissue part 12 has been treated for implantation, the second tissue part 14 may be removed from the embedding material 40. By way of example and without limitation, the embedding material 40 may be peeled from the implant 10, washed or dissolved using suitable solvents or solutions that are substantially non-reactive with the tissue implant in total or in part (e.g., water, salt solution, alcohol, chloroform, acetone, MeCl2), melted off the implant 10 using low heating that is not detrimental to the implant, combinations thereof, or other ways known to those of ordinary skill in the art.

Depending on the specific application, and as shown in FIG. 2B, the second tissue part 14 may likewise be treated for implantation using a second preparation method. In a manner similar to that described above, the first tissue part 12, having now been prepared, may be substantially protected from exposure to the second preparation method. To this end, the first tissue part 12 may be embedded in an embedding material 44, which may be the same or different from embedding material 40 depending on the tissue type, the preparation method being used, and/or other factors. The second tissue part 14 is positioned within the cavity 32 external to the embedding material 44. A processing reagent 42, such as acidic solution, base solution, detergents, organic solvent and enzyme solution, etc., may flow through the cavity 32 via the fluid inlet and outlet 34, 36, respectively, so as to treat the second tissue part 14. Again, the processing reagent 46 may be contained or held in cavity 32 without flow-through capability and the inlet and outlet omitted. As noted above, the embedding material 44 protects the first tissue part 12 and substantially prevents exposure of the first tissue part 12 to the second preparation method. Thus, for example, the processing reagent 46 is not capable of substantially penetrating the embedding material 44 so as to reach and/or react with the first tissue part 12. After the second tissue part 14 has been treated for implantation, the first tissue part 12 may be removed from the embedding material 44 as described above.

While the embedding materials 40, 44 were described above as being substantially non-reactive with the second tissue part 14 and first tissue part 12, respectively, the invention is not so limited. In particular, the embedding materials 40, 44 may include one or more components that treat the tissue parts embedded therein. Thus, for example, the embedding material 40 may include a component that treats the second tissue part 14 as the first tissue part 12 is being processed by the first preparation method. Likewise, the embedding material 44 may include a component that treats the first tissue part 12 as the second tissue part 14 is being processed by the second preparation method.

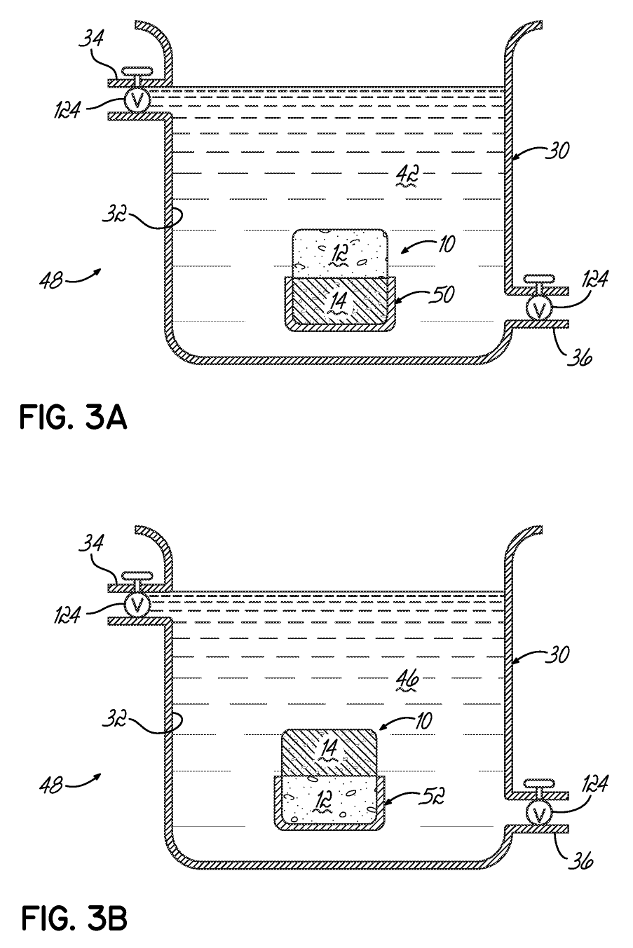

FIGS. 3A and 3B, in which like reference numerals refer to like features in FIGS. 2A and 2B, illustrate another embodiment wherein the tissue parts 12, 14 of a tissue implant 10 are substantially protected from the preparation methods through a coating technique. To this end, an apparatus 48 for preparing a tissue implant 10 includes a container 30 having an interior cavity 32 and a fluid inlet 34 and outlet 36 in communication with the interior cavity 32. The container 30 may optionally include a top (not shown) for closing off the interior cavity 32. As shown in FIG. 3A, tissue implant 10 is positioned in interior cavity 32 for treating the first tissue part 12. Prior to insertion of the tissue implant 10 in cavity 32, however, the second tissue part 14 may be coated with at least one coating material 50 that substantially prevents exposure of the second tissue part 14 to the first preparation method.

In one embodiment, the coating material 50 may be a solid or semi-solid material such as, for example and without limitation, wax, polystyrene, poly(methyl methacrylate), combinations thereof, and other materials known to those of ordinary skill in the art. As explained below, any suitable coating material 50 may be used which of itself is substantially non-reactive with the second tissue part 14 and which substantially prevents the second tissue part 14 from exposure to the first preparation method. In addition, the coating material 50 should be relatively easy to remove from the second tissue part 14. Moreover, the coating material 50 may be applied to the second tissue part 14 in any manner known to those of ordinary skill in the art. For example and without limitation, the coating material 50 may be sprayed on, brushed on, applied by dipping, etc. The coating material may have a typical thickness of 1 nm to 10 mm, but preferably less than 1 mm.

With the second tissue part 14 coated by the material 50 and the tissue implant 10 positioned within the cavity 32, the first tissue part 12 may be processed using a first preparation method. For example, a processing reagent 42, such as acidic solution, base solution, detergents, organic solvent and enzyme solution, etc., may flow through the cavity 32 via the fluid inlet and outlet 34, 36, respectively, so as to treat the first tissue part 12. While container 30 includes an inlet and outlet that permits a fluid to flow through the cavity 32, the invention is not so limited as the inlet and outlet may be omitted and the container 30 adapted to hold or contain processing reagent 42 without flow-through capability. As noted above, the coating material 50 protects the second tissue part 14 and substantially prevents exposure of the second tissue part 14 to the first preparation method. Thus, for example, the processing reagent 42 is not capable of substantially penetrating the coating material 50 so as to reach and/or react with the second tissue part 14. After the first tissue part 12 has been treated for implantation, the coating material 50 may be removed from the second tissue part 14. By way of example and without limitation, the coating material 50 may be peeled from the implant 10, washed or dissolved using suitable solvents or solutions that are substantially non-reactive with the tissue implant in total or in part (e.g., water, salt solution, alcohol, chloroform, acetone, MeCl2), melted off the implant 10 using low heating that is not detrimental to the implant, combinations thereof, or other ways known to those of ordinary skill in the art.

Depending on the specific application, and as shown in FIG. 3B, the second tissue part 14 may likewise be treated for implantation using a second preparation method. In a manner similar to that described above, the first tissue part 12, having now been prepared, may be substantially protected from exposure to the second preparation method. To this end, the first tissue part 12 may be coated by coating material 52, which may be the same or different from coating material 50 depending on the tissue type, the preparation method being used, and/or other factors. The tissue implant 10 is positioned within the cavity 32. A processing reagent 46, such as acidic solution, base solution, detergents, organic solvent and enzyme solution, etc., may flow through the cavity 32 via the fluid inlet and outlet 34, 36, respectively, so as to treat the second tissue part 14. Again, the processing reagent 46 may be contained or held in cavity 32 without flow-through capability and the inlet and outlet omitted. As noted above, the coating material 52 protects the first tissue part 12 and substantially prevents exposure of the first tissue part 12 to the second preparation method. Thus, for example, the processing reagent 46 is not capable of substantially penetrating the coating material 52 so as to reach and/or react with the first tissue part 12. After the second tissue part 14 has been treated for implantation, the first tissue part 12 may be removed from the coating material 52 as described above.

While the coatings 50, 52 were described above as being substantially non-reactive with the second tissue part 14 and first tissue part 12, respectively, the invention is not so limited. In particular, the coatings 50, 52 may include one or more components that treat the tissue parts that are coated. Thus, for example, the coating material 50 may include a component that treats the second tissue part 14 as the first tissue part 12 is being processed by the first preparation method. Likewise, the coating 52 may include a component that treats the first tissue part 12 as the second tissue part 14 is being processed by the second preparation method.

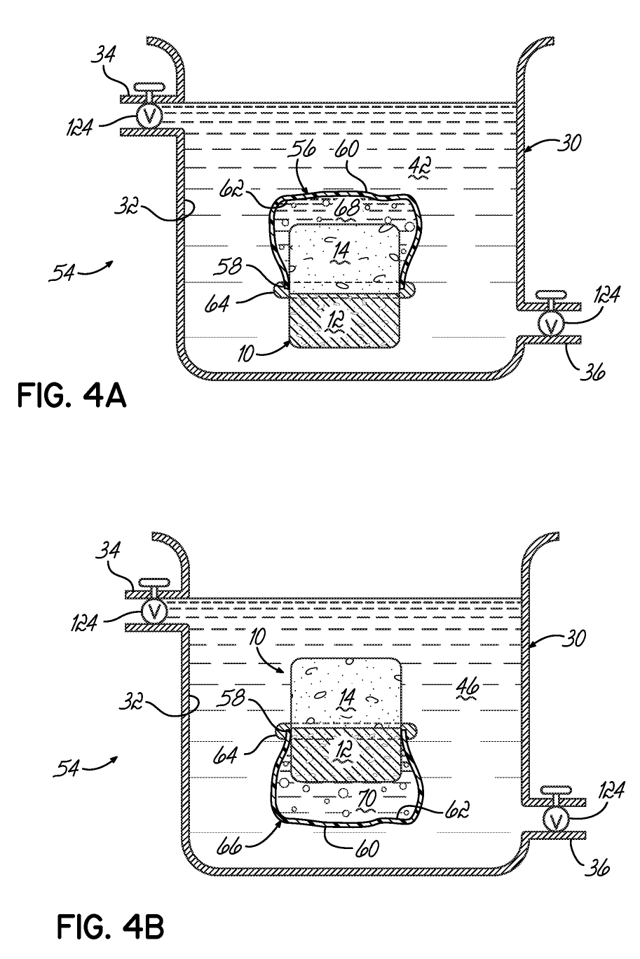

FIGS. 4A and 4B, in which like reference numerals refer to like features in FIGS. 2A and 2B, illustrate another embodiment wherein the tissue parts 12, 14 of a tissue implant 10 are substantially protected from the preparation methods through a covering technique. To this end, an apparatus 54 for preparing tissue implant 10 includes a container 30 having an interior cavity 32 and a fluid inlet 34 and outlet 36 in communication with the interior cavity 32. The container 30 may optionally include a top (not shown) for closing off the interior cavity 32. As shown in FIG. 4A, tissue implant 10 is positioned in interior cavity 32 for treating the first tissue part 12. Prior to insertion of the tissue implant 10 in cavity 32, however, the second tissue part 14 may be covered with a cover 56 that substantially prevents exposure of the second tissue part 14 to the first preparation method.

In one embodiment, the cover 56 includes a first open end 58 and a second closed end 60 that defines an interior cavity 62. The cover 56 may be made from a material that is substantially non-reactive with the second tissue part 14 and which substantially prevents the second tissue part 14 from exposure to the first preparation method. For example and without limitation, the cover 56 may be formed from a material that is resistant to the aforementioned processing reagents or impermeable by big molecules (e.g., enzymes) or hydrophobic. The second tissue part 14 may be inserted into the cover 56 via the open end 58 and the open end 58 closed about the tissue implant 10 to form a seal thereat. For example, a clip 64, such as an elastic band, may be used to secure the cover 56 to the tissue implant 10 and form a seal along open end 58. Alternatively, a sealant, such as polyurethane or silicone, may be used to form the seal along open end 58. Those of ordinary skill in the art will recognize other ways to secure the cover 56 to the tissue implant 10 and form a seal therewith. The clip, sealant or other sealing elements also may be substantially non-reactive with the tissue and/or processing reagent(s) with which it is used.

With the second tissue part 14 disposed in the interior cavity 62 of cover 56 and the first tissue part 12 external to the cover 56, the tissue implant 10 may be positioned within the cavity 32 and the first tissue part 12 processed using a first preparation method. For example, a processing reagent 42, such as acidic solution, base solution, detergents, organic solvent and enzyme solution, etc., may flow through the cavity 32 via the fluid inlet and outlet 34, 36, respectively, so as to treat the first tissue part 12. While container 30 includes an inlet and outlet that permits a fluid to flow through the cavity 32, the invention is not so limited as the inlet and outlet may be omitted and the container 30 adapted to hold or contain processing reagent 42 without flow-through capability. As noted above, the cover 56 protects the second tissue part 14 and substantially prevents exposure of the second tissue part 14 to the first preparation method. Thus, for example, the processing reagent 42 is not capable of substantially penetrating the cover 56 so as to reach and/or react with the second tissue part 14. After the first tissue part 12 has been treated for implantation, the cover 56 may be removed from the second tissue part 14 by, for example, removing the clip 64 or removing the sealant from the open end 58.

Depending on the specific application, and as shown in FIG. 4B, the second tissue part 14 may likewise be treated for implantation using a second preparation method. In a manner similar to that described above, the first tissue part 12, having now been prepared, may be substantially protected from exposure to the second preparation method. To this end, the first tissue part 12 may be disposed in the interior of a cover 66, which may be made from the same or different material as cover 56 depending on the tissue type, the preparation method being used, and/or other factors. The tissue implant 10 is positioned within the cavity 32. A processing reagent 46, such as acidic solution, base solution, detergents, organic solvent and enzyme solution, etc., may flow through the cavity 32 via the fluid inlet and outlet 34, 36, respectively, so as to treat the second tissue part 14. Again, the processing reagent 46 may be contained or held in cavity 32 without flow-through capability and the inlet and outlet omitted. As noted above, the cover 66 protects the first tissue part 12 and substantially prevents exposure of the first tissue part 12 to the second preparation method. Thus, for example, the processing reagent 46 is not capable of substantially penetrating the cover 66 so as to reach the first tissue part 12. After the second tissue part 14 has been treated for implantation, the first tissue part 12 may be removed from the cover 66.

In one embodiment, the interior cavity 62 of the cover 56 (FIG. 4A) may contain at least one preparatory solution 68 for treating the second tissue part 14 (e.g., prior to, during and/or after the first tissue part 12 being treated by the first preparation method as described above). For example, the cavity 62 of cover 56 may contain humidified air, water, saline, or PBS, etc. The preparatory solution 68 may protect the second tissue part 14 from dehydration, hyper-hydration, biological degradation, loss of structural or biomechanical integrity, etc. Additionally, the preparatory solution 68 may operate to process the second tissue part 14 for implantation. For example, treatment of the second tissue part 14 may be achieved by the preparatory solution 68 without any further treatment, i.e., the second preparation method on the second tissue part as described above is not performed. Alternatively, the preparatory solution 68 may be used in combination with the second preparation method as described above. Moreover, the interior cavity of cover 66 (FIG. 4B) may contain at least one preparatory solution 70 as the second tissue part 14 is being treated by the second preparation method. In one embodiment, one or both of the covers 56, 66 are impermeable to components of the first and second reagents. Alternatively, however, one or both of the covers 56, 66 may be selectively permeable to at least one component of the first and/or second reagents to perform some function on the tissue part disposed in the interior cavity of the cover. Such selective permeability would allow, for example, the first and second tissue parts to be exposed to differing concentrations of the same reagent.

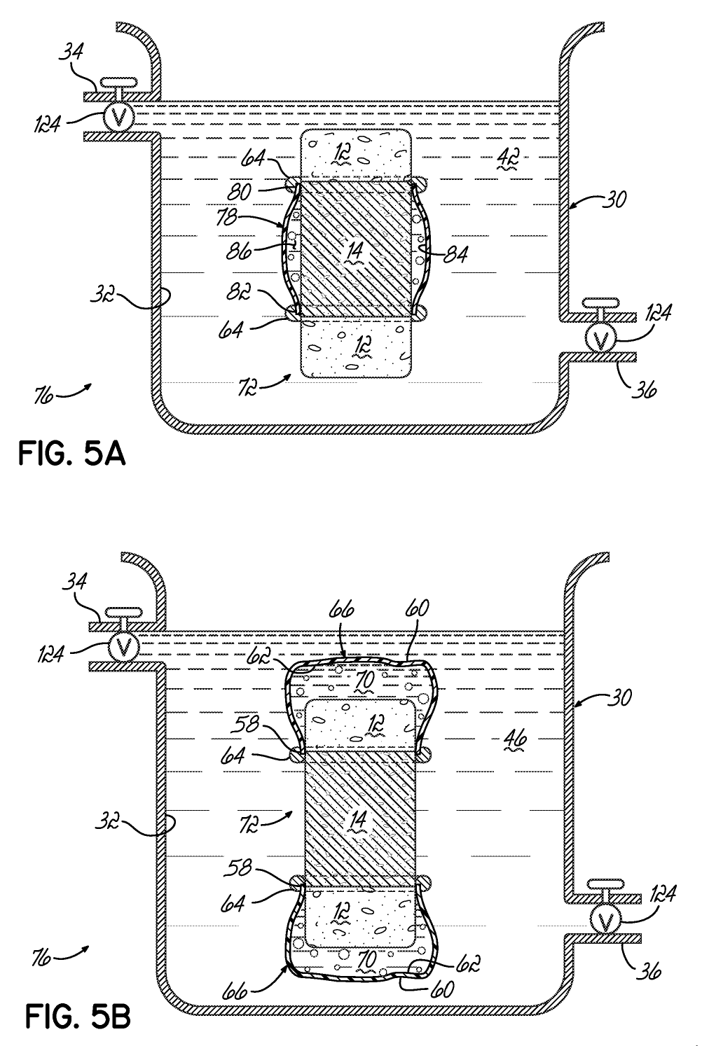

FIGS. 5A and 5B, in which like reference numerals refer to like features in FIGS. 2A, 2B, 4A and 4B illustrate another embodiment wherein the tissue implant 72 includes first and second tissue parts 12, 14, wherein the second tissue part 14 is disposed between two portions of the first tissue part 12. In this embodiment, the second tissue part 12 may be substantially protected using a modified covering technique as that described above. To this end, an apparatus 76 for preparing a tissue implant 72 includes a container 30 having an interior cavity 32 and a fluid inlet 34 and outlet 36 in communication with the interior cavity 32. The container 30 may optionally include a top (not shown) for closing off the interior cavity 32. As shown in FIG. 5A, tissue implant 72 is positioned in interior cavity 32 for treating the first tissue parts 12 on either side of second tissue part 14. Prior to insertion of the tissue implant 72 in cavity 32, however, the second tissue part 14 may be covered with a cover 78 that substantially prevents exposure of the second tissue part 14 to the first preparation method.

In one embodiment, the cover 78 includes a first open end 80 and a second open end 82 that define an interior passage 84. The cover 78 may be made from a material that is substantially non-reactive with the second tissue part 14 and which substantially prevents the second tissue part 14 from exposure to the first preparation method. For example and without limitation, the cover 78 may be formed from a material that is resistant to the aforementioned processing reagents or impermeable by big molecules (e.g., enzymes) or hydrophobic. The second tissue part 14 may be inserted in the interior passage 84 of the cover 78 via one of the open ends 80, 82 and each of the open ends 80, 82 closed about the tissue implant 72 to form a seal thereat. For example, a clip 64 may be used to secure the cover 78 to the tissue implant 72 and form a seal along open ends 80, 82. Alternatively, a sealant, such as polyurethane or silicone, may be used to form the seal along open ends 80, 82. Those of ordinary skill in the art will recognize other ways to secure the cover 78 to the tissue implant 10 and form a seal therewith. The clip, sealant or other sealing elements may be substantially non-reactive with both the tissue and processing reagent(s) with which they are used.

With the second tissue part 14 disposed in the interior passage 84 of cover 78 and the first tissue parts 12 external to the cover 78 and positioned within the cavity 32, the first tissue parts 12 may be processed using a first preparation method. For example, a processing reagent 42, such as acidic solution, base solution, detergents, organic solvent and enzyme solution, etc., may flow through the cavity 32 via the fluid inlet and outlet 34, 36, respectively, so as to treat the first tissue part 12. While container 30 includes an inlet and outlet that permits a fluid to flow through the cavity 32, the invention is not so limited as the inlet and outlet may be omitted and the container 30 adapted to hold or contain processing reagent 42 without flow-through capability. As noted above, the cover 78 protects the second tissue part 14 and substantially prevents exposure of the second tissue part 14 to the first preparation method. Thus, for example, the processing reagent 42 is not capable of substantially penetrating the cover 78 so as to reach and/or react with the second tissue part 14. After the first tissue part 12 has been treated for implantation, the cover 78 may be removed from the second tissue part 14 by, for example, removing the clip 64 or removing the sealant from the open ends 80, 82.

Depending on the specific application, and as shown in FIG. 5B, the second tissue part 14 may likewise be treated for implantation using a second preparation method. In a manner similar to that described above in reference to FIG. 4B, the first tissue parts 12, having now been prepared, may be substantially protected from exposure to the second preparation method. To this end, the first tissue parts 12 may each be disposed in the interior of a cover 66 as described above, and the second tissue part 14 positioned within the cavity 32. A processing reagent 46, such as acidic solution, base solution, detergents, organic solvent and enzyme solution, etc., may flow through the cavity 32 via the fluid inlet and outlet 34, 36, respectively, so as to treat the second tissue part 14. Again, the processing reagent 46 may be contained or held in cavity 32 without flow-through capability and the inlet and outlet omitted. As noted above, each of the covers 66 protects the first tissue part 12 and substantially prevents exposure of the first tissue part 12 to the second preparation method. Thus, for example, the processing reagent 46 is not capable of substantially penetrating the covers 66 so as to reach the first tissue part 12. After the second tissue part 14 has been treated for implantation, the first tissue parts 12 may be removed from the covers 66.

In one embodiment, the interior passage 84 of the cover 78 (FIG. 5A) may contain at least one preparatory solution 86 for treating the second tissue part 14 as the first tissue parts 12 are being treated by the first preparation method as described above. For example, the passage 84 of cover 78 may contain humidified air, water, saline, or PBS, etc. The preparatory solution 86 may protect the second tissue part 14 from dehydration, hyper-hydration, biological degradation, loss of structural or biomechanical integrity, etc. Additionally, the preparatory solution 86 may operate to process the second tissue part 14 for implantation. For example, treatment of the second tissue part 14 may be achieved by the preparatory solution 86 without any further treatment, i.e., the second preparation method on the second tissue part 14 as described above is not performed. Alternatively, the preparatory solution 86 may be used in combination with the second preparation method as described above. Moreover, the interior cavity of covers 66 (FIG. 5B) may contain at least one preparatory solution 70 as the second tissue part 14 is being treated by the second preparation method, as described above in reference to FIG. 4B. In one embodiment, the cover 78 is impermeable to components of the first reagent. Alternatively, however, the cover 78 may be selectively permeable to at least one component of the first reagent to perform some treatment function on the tissue part disposed in the interior passage 84 of the cover 78.

Though the tri-section tissue implant 72 described above was differentially processed using a covering technique, such tri-section tissue implants, or tissue implants with even more sections, may be differentially processed using any of the techniques described herein (e.g., embedding, covering, coating, and/or isolating).

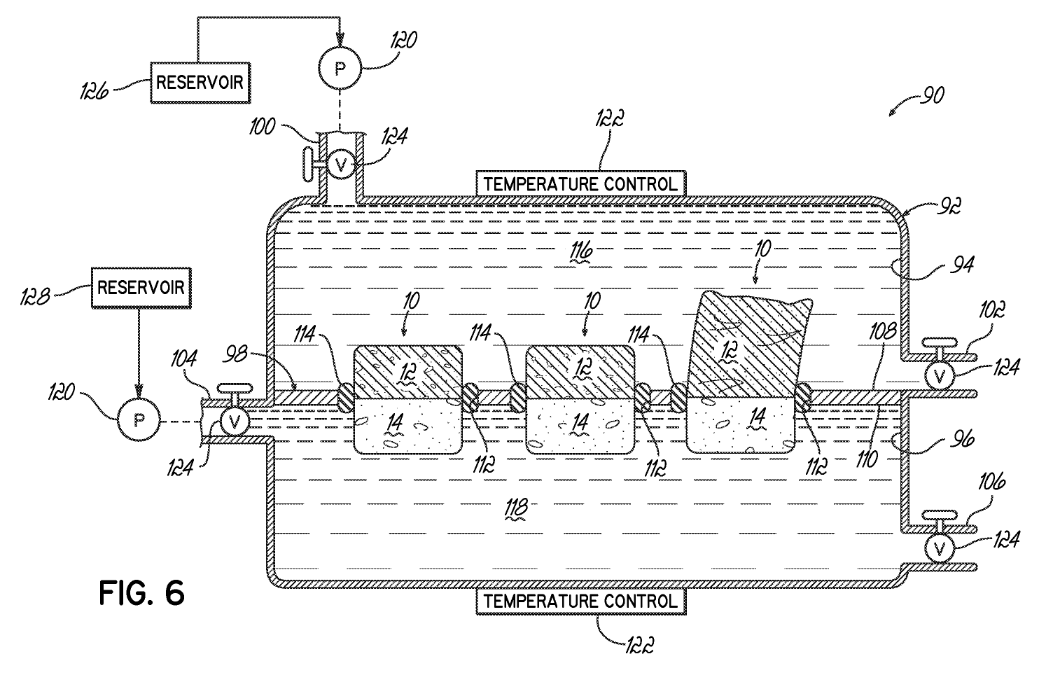

FIG. 6 illustrates another embodiment wherein the tissue parts 12, 14 of a tissue implant 10 are substantially protected from the preparation methods using a multi-chambered apparatus wherein the chambers are physically separated and substantially isolated (e.g., with respect to various treatment conditions) from each other. To this end, an apparatus 90 for preparing a tissue implant 10 includes an enclosure 92 having a first interior chamber 94 separated from a second interior chamber 96 by a partition member 98. The first interior chamber 94 includes a fluid inlet 100 and outlet 102 in communication with the first interior chamber 94. In a similar manner, the second interior chamber 96 likewise includes a fluid inlet 104 and outlet 106 in communication with the second interior chamber 96. As shown in FIG. 6, the partition member 98 includes a first surface 108 that forms part of the boundary of the first chamber 94, and a second surface 110 that forms part of the boundary of the second chamber 96. The partition member 98 further includes at least one aperture 112 (three shown) therethrough and extending between the first and second surfaces 108, 110. The tissue implant 10 is configured to be coupled to the partition member 98 so that a substantial portion of the first tissue part 12 is within the first chamber 94 and a substantial portion of the second tissue part 14 is within the second chamber 96. To this end, the aperture 112 may include a sealing ring, generally shown at 114, that not only secures the tissue implant 10 to the partition member 98, but also forms a seal between the first and second chambers 94, 96. For example, in one embodiment, the sealing ring 114 may be a rubber O-ring capable of resiliently retaining the tissue implant 10 therein and forming a seal with the tissue implant 10. Alternatively, the sealing ring may be a wax or other adhesive substance capable of retaining the tissue implant 10 in partition member 98 and forming a seal with implant 10. Those of ordinary skill in the art will appreciate other materials which may be used as sealing ring 114. The sealing ring may preferably be substantially non-reactive with the tissue and processing reagent(s) which contact the sealing ring.

With the first tissue part 12 substantially disposed in the first chamber 94 and the second tissue part 14 substantially disposed in the second chamber 96, the first tissue part 12 may be processed using a first preparation method and the second tissue part 14 may be processed using a second preparation method. For example, a first processing reagent 116, such as acidic solution, base solution, detergents, organic solvent and enzyme solution, etc., may flow through the first chamber 94 via the fluid inlet and outlet 100, 102, respectively, so as to treat the first tissue part 12. In a similar manner, a second processing reagent 118, such as acidic solution, base solution, detergents, organic solvent and enzyme solution, etc., may flow through the second chamber 96 via the fluid inlet and outlet 104, 106, respectively, so as to treat the second tissue part 14. Flow of the first and second processing reagents through the first and second chambers may be simultaneous or sequential, continuous or discontinuous in various embodiments. While each of the chambers 94, 96 includes an inlet and outlet that permits a fluid to flow through the chambers, the invention is not so limited as the inlets and outlets may be omitted and the chambers adapted to hold or contain processing reagents 116, 118 without flow-through capability. As noted above, the chambers 94, 96 are sealed at the partition member 98 so that the first tissue part 12 is not substantially exposed to the second preparation method and the second tissue part is not substantially exposed to the first preparation method. Such an apparatus 90 allows the first and second tissue parts 12, 14 to be prepared, at least in part, essentially at the same time. Those of ordinary skill in the art will recognize, however, that the tissue implant 10 may be processed sequentially in apparatus 90.

The apparatus 90 may further include additional elements for implementing the first and second preparation methods. For example, the first and/or second preparation methods may include varying or otherwise manipulating the pressure, temperature, concentration, and/or flow rate of the processing reagents 116, 118 through the chambers. To this end, the apparatus 90 may include a pump 120 associated with at least one and preferably both of the inlets 100, 104 to the first and second chambers 94, 96. The pump(s) 120 are capable of regulating the pressure of the processing reagents 116, 118 in the first and/or second chambers 94, 96. The apparatus 90 may also include a thermal control unit 122 associated with at least one and preferably both of the chambers 94, 96 for controlling the temperature of the first and/or second processing reagents 116, 118. The thermal control unit 22 includes a heat-generating element (not shown), such as a heating coil, for selectively heating the reagents in the first and/or second chambers 94, 96. The thermal control unit 22 may also include a temperature sensor (not shown) for monitoring the temperature of the first and/or second reagents and controlling the heat-generating element to maintain a desired temperature. The thermal control unit 22 may further include a capacity to cool the processing reagents in the first and second chambers 94, 96. Such thermal control units 22 are generally well known in the art. When temperature is manipulated as an aspect in either the first or second preparation method, the partition member 98 and/or sealing rings 114 may be made of a low thermally conductive material (i.e., an insulator) so as to minimize any heat transfer between the first and second chambers 94, 96.

To control the flow of the processing reagents 116, 118 through the chambers 94, 96 the apparatus 90 may further include a valve 124 coupled to at least one of the inlet or outlet of each of the chambers 94, 96. By adjusting the valves 124 between opened and closed positions, the flow rate of the processing reagents 116, 118 may be varied depending on the specific preparation method being used on the tissue parts. Such valves are also well known in the art. Moreover, as noted above, the concentration of components of the processing reagents 116, 118 may be varied depending on the specific preparation method being used. Accordingly, the fluid inlets 100, 104 to the chambers 94, 96 may be in fluid communication with a fluid reservoir 126, 128, respectively, that holds the processing reagents 116, 118 with the desired components and at the desired concentrations, or at bulk concentrations for achieving desired dilutions with water buffer solutions, alcohol, or other diluents.

While the present invention has been illustrated by a description of various preferred embodiments and while these embodiments have been described in detail, it is not the intention of the inventors to restrict or in any way limit the scope of the appended claims to such detail. While the embodiments described herein show a tissue implant having a first and second tissue part, the invention is not so limited. Embodiments of the invention may be used to prepare tissue implants with more than two tissue parts, with each part having a corresponding preparation method. Those of ordinary skill in the art will recognize how to modify the embodiments described herein to accommodate additional tissue parts. For example, the enclosure shown in FIG. 6 can be modified to have more than two chambers with each of the chambers separated by a partition member and a substantial part of a tissue part being disposed in each of the chambers. Moreover, the embedding materials, coating materials, and covers may be used in various combinations to accommodate tissue implants with more than two tissue parts. It should be realized that the first and second processing methods may be substantially the same but differ only by the duration of exposure, temperature, or other limited change. For example, the second tissue part could be protected during the initial exposure of the first tissue part to a treatment process, and then subsequently both tissue parts treated simultaneously under the same conditions.

Furthermore, the various techniques, i.e., embedding, covering, coating, and isolation, may be used in various combinations to prepare a tissue implant. Also, while many of the apparatus shown and described herein illustrates only one tissue implant being processed, those of ordinary skill in the art will recognize that the apparatus may be configured to hold multiple tissue implants. Thus, additional advantages and modifications will readily appear to those skilled in the art. The various features of the invention may be used alone or in numerous combinations depending on the needs and preferences of the user.

What is claimed is:

1. A method of preparing an implantable biological device comprising at least a first biological tissue part and a second biological tissue part, the method comprising exposing the first biological tissue part to a first preparation method, and simultaneously substantially preventing exposure of the second biological tissue part to the first preparation method by embedding the second biological tissue part in a material that substantially prevents exposure of the second biological tissue part to the first preparation method.

2. The method of claim 1 wherein the embedding material is selected from the group consisting of gases, liquids, solids, semi-solids, and combinations thereof.

3. The method of claim 1 wherein the embedding material is selected from the group consisting of wax, polystyrene, poly(methyl methacrylate), poly-alginate, agarose gel, and combinations thereof.

4. The method of claim 1 further comprising exposing the second biological tissue part to a second preparation method.

5. The method of claim 4 further comprising simultaneously substantially preventing exposure of the first biological tissue part to the second preparation method by embedding the first biological tissue part in a material that substantially prevents exposure of the first biological tissue part to the second preparation method.

6. The method of claim 1 wherein the first and second biological tissue parts are sections of a homogeneous tissue.

7. The method of claim 1 wherein the first biological tissue part is bone and the second biological tissue part is selected from the group consisting of cartilage, tendon, ligament, meniscus, periosteum, muscle, and combinations thereof.

8. A tissue implant prepared according to the method of claim 1.

9. A method of preparing an implantable biological device comprising at least a first biological tissue part and a second biological tissue part, the method comprising exposing the first biological tissue part to a first preparation method, and simultaneously substantially preventing exposure of the second biological tissue part to the first preparation method by coating the second biological tissue part with a material that substantially prevents exposure of the second biological tissue part to the first preparation method.

10. The method of claim 9 wherein the coating material is selected from the group consisting of wax, polystyrene, poly(methyl methacrylate), and combinations thereof.

11. The method of claim 9 further comprising exposing the second biological tissue part to a second preparation method.

12. The method of claim 11 further comprising simultaneously substantially preventing exposure of the first biological tissue part to the second preparation method by coating the first biological tissue part with a material that substantially prevents exposure of the first biological tissue part to the second preparation method.

13. The method of claim 9 wherein the first and second biological tissue parts are sections of a homogeneous tissue.

14. The method of claim 9 wherein the first biological tissue part is bone and the second biological tissue part is selected from the group consisting of cartilage, tendon, ligament, meniscus, periosteum, muscle, and combinations thereof.

15. The method of claim 9 wherein the first biological tissue part is one of the red or white zones of the meniscus and the second biological tissue part is the remaining portion of the meniscus.

16. A tissue implant prepared according to the method of claim 9.

17. A method of preparing an implantable biological device comprising at least a first biological tissue part and a second biological tissue part, the method comprising exposing the first biological tissue part to a first preparation method, and simultaneously substantially preventing exposure of the second biological tissue part to the first preparation method by covering the second biological tissue part with a cover that substantially prevents exposure of the second biological tissue part by the first preparation method, the cover including a first open end and a second closed end to define an interior cavity, the method further comprising positioning the second biological tissue part in the interior cavity of the cover, and sealing the first end of the cover to the tissue part to substantially enclose the second tissue part inside the cover.

18. The method of claim 17 further comprising maintaining at least one preparation solution in the interior cavity of the cover.

19. The method of claim 17 wherein the cover is selectively permeable to at least one component of a reagent used in the first preparation method.

20. The method of claim 17 wherein the cover is impermeable.

21. The method of claim 17 wherein the first and second biological tissue parts are sections of homogeneous tissue.

22. A method of preparing an implantable biological device comprising at least a first biological tissue part and a second biological tissue part, the method comprising exposing the first biological tissue part to a first preparation method, and simultaneously substantially preventing exposure of the second biological tissue part to the first preparation method by covering the second biological tissue part with a cover that substantially prevents exposure of the second biological tissue part by the first preparation method, the cover including a first open end and a second open end to define an interior passage, the method further comprising positioning the second tissue biological part in the interior passage of the cover, and sealing the first and second ends of the cover to the biological tissue part to substantially enclose the second tissue part inside the cover.

23. The method of claim 22 further comprising maintaining at least one preparation solution in the interior passage of the cover.

24. The method of claim 22 wherein the cover is selectively permeable to at least one component of a reagent used in the first preparation method.

25. The method of claim 22 wherein the cover is impermeable.

26. The method of claim 22 wherein the first and second biological tissue parts are sections of homogeneous tissue.

27. A method of preparing an implantable biological device comprising at least a first biological tissue part and a second biological tissue part, the method comprising exposing the first biological tissue part to a first preparation method, and simultaneously substantially preventing exposure of the second biological tissue part to the first preparation method by positioning the first biological tissue part in a first chamber of an enclosure, and positioning the second biological tissue part in a second chamber of the enclosure that is substantially isolated from the first chamber.

28. The method of claim 27 further comprising exposing the first biological tissue part to a first processing reagent within the first chamber of the enclosure, and exposing the second biological tissue part to a second processing reagent within the second chamber of the enclosure.

29. The method of claim 28 further comprising varying one of the temperature, pressure, and concentration of at least one of the first or second reagents.

30. The method of claim 27 further comprising thermally isolating the first chamber of the enclosure from the second chamber of the enclosure.

31. The method of claim 27 wherein the first and second biological tissue parts are sections of homogeneous tissue.

32. The method of claim 27 wherein the first biological tissue part is bone and the second biological tissue part is selected from the group consisting of cartilage, tendon, ligament, meniscus, periosteum, muscle, and combinations thereof.

33. A tissue implant prepared according to the method of claim 27.

34. An apparatus for preparing an implantable biologic device, the apparatus comprising:
an enclosure having a first chamber and a second chamber separated by a partition member that substantially isolates the chambers from one another; and
at least one aperture in the partition member adapted to receive an implantable biological tissue, the tissue having a first tissue part and a second tissue part, the implantable biological tissue adapted to be received in the aperture so that a substantial portion of the first tissue part is within the first chamber and a substantial portion of the second tissue part is within the second chamber.

35. The apparatus of claim 34 further comprising a first chamber inlet and outlet for exposing the first tissue part to a first processing reagent, and a second chamber inlet and outlet for exposing the second tissue part to a second processing reagent.

36. The apparatus of claim 35 further comprising a thermal control unit thermally coupled to at least one of the first or second chambers and adapted to control the temperature of the respective first and/or second reagent.

37. A method of preparing an implantable biological device comprising at least a first biological tissue part and a second biological tissue part, the method comprising exposing the first biological tissue part to a first processing reagent, and simultaneously substantially preventing exposure of the second biological tissue part to the first processing reagent.
